(19) United States
(12) Patent Application Publication
(10) Pub. No.: US 2005/0033406 A1
(43) Pub. Date: Feb. 10, 2005

(54) Branch vessel stent and graft

(76) Inventors: William H. Barnhart, Iowa City, IA (US); Jamal J. Hoballah, Coralville, IA (US)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 10/891,917

(22) Filed: Jul. 15, 2004

Related U.S. Application Data
(60) Provisional application No. 60/487,428, filed on Jul. 15, 2003.

(51) Int. Cl. A61F 2/06

(52) U.S. Cl. 623/1.13

(57) ABSTRACT

Endovascular stents and grafts for repairing portions of anatomical vessels are provided. The stent grafts may be attached to a graft positionable at a junction where a vessel divides or branches with at least one other vessel for repair of an aneurysm in the region of the junction.

This application claims the benefit of U.S. Provisional Application No. 60/487,428, filed Jul. 15, 2003, the entirety of which is hereby incorporated by reference.

### FIELD OF THE INVENTION

The present invention relates generally to devices and methods for repairing aneurysms, and more particularly to percutaneous and/or intraluminal devices having side branches extending therefrom for repairing aneurysms.

### BACKGROUND

An aneurysm is an abnormal dilation of a layer or layers of an arterial wall. Aortic aneurysms involve one or more of the various regions of the aorta 10 as shown in FIG. 1. The aorta 10 may be divided into several regions including the ascending aorta 12, the aortic arch 14, the descending aorta 16 and the abdominal aorta 18. Various vessels such as the renal arteries 20a and 20b branch off of the aorta to supply the organs of the body such as the kidneys 21a and 21b with blood. The distal end of the aorta bifurcates into the iliac arteries 22a and 22b which supply the legs with blood.

The various types of aortic aneurysms may be classified on the basis of the region(s) of aneurysmic involvement and include thoracic, thoracoabdominal and abdominal. Thoracic aortic aneurysms involve the ascending thoracic aorta 12 and/or the aortic arch 14 and associated branch arteries such as the subclavian arteries (not shown). Thoracoabdominal aortic aneurysms involve the descending thoracic aorta 16 and associated branch arteries and/or the suprarenal abdominal aorta 18 and associated branch arteries such as the renal 20a and 20b, superior mesenteric (not shown) and intercostal (not shown) arteries. Abdominal aortic aneurysms involve the pararenal aorta and the associated branch arteries such as the renal arteries 20a and 20b as well as aneurysms involving the infrarenal aorta with or without iliac involvement.

Traditional surgical repair of aortic aneurysms is not always a viable option as many patients diagnosed with aortic aneurysms are in relatively poor health and are considered poor surgical risks. Additionally, the traditional surgical approach to repair of aortic aneurysms requires cross-clamping of the aorta above the aneurysm, which can result in ischemic damage to organs supplied with blood by vessels inferior to the aneurysm or other undesired results. Nonetheless, if allowed to remain untreated, a substantial percentage of aortic aneurysms may ultimately rupture, with catastrophic consequences to the patient.

One alternative to the traditional surgical methods of repairing aneurysms involves the technique of endovascular grafting. Endovascular grafting is a relatively noninvasive method using a body introduction device to place a tubular graft within the lumen of a blood vessel. In certain cardiovascular applications of the technique such as that shown in FIG. 2, an endovascular graft 32 is implanted within an aneurysmic segment of a blood vessel 30 to form a prosthetic flow conduit through the aneurysm. The endovascular graft effectively isolates the weakened portion of the blood vessel wall from the hemodynamic forces and pressures of the flowing blood.

In general, endovascular grafts typically comprise a tube or sheath 24 of a pliable covering material in combination with one or more anchoring components 26. Typical covering materials include expanded polytetrafluoroethylene (ePTFE) and woven polyester. Anchoring component include stents, frames, wire rings, hooks, barbs and/or clips which operate to hold the tubular graft in the desired position within the blood vessel. Typically, the anchoring component is formed of an expandable stent or frame which is either incorporated into the body of the tubular graft or formed separately from the graft and deployed within the graft lumen, although other types of expandable stents or frames may be utilized. The stent is designed to exert outwardly directed radial pressure against the surrounding blood vessel wall when expanded to frictionally hold the graft in place.

Endovascular grafts incorporating radially expandable graft anchoring components are initially deployed in a collapsed configuration which is sufficiently compact to allow the graft to be transluminally advanced through the vasculature until it reaches the implantation site. Once the implantation site is reached, the graft is expanded to an expanded configuration which is large enough to exert the desired pressure against the blood vessel wall. In some embodiments, hooks, barbs, or other projections on the graft anchoring component will insert into the wall of the blood vessel to ensure that the graft will be tightly held in the desired position.

Expandable graft components are generally classifiable as either self-expanding or pressure-expandable. Self-expanding graft components are usually formed of a resilient form-returning material that returns to a previous form, or shape, when a particular event transpires, such as removal of a constraining device associated with the body introduction device or increase in temperature. An example form returning material is a spring metal, such as spring steel. A particular type of form-returning material useful in forming stents is a shape memory alloy, which, after being deformed, can recover its original shape when heated. An example shape memory alloy is Nickel-Titanium (“Nitinol”). Typically, the expandable graft components automatically expand from a radially collapsed configuration to a radially expanded configuration when relieved of a surrounding constraint, such as a surrounding tubular sheath or catheter wall.

Pressure-expandable graft components are typically formed of malleable wire or other plastically deformable material which will deform to an expanded configuration in response to the exertion of outwardly directed pressure. Typically this outward pressure is provided by inflation of a balloon catheter or actuation of another pressure-exerting apparatus which is positioned within the graft components.

Depending on which regions of the aorta are involved, the aneurysm may extend into bifurcated areas of the aorta, such as where the inferior aorta bifurcates into the iliac arteries, or segments of the aorta from which smaller arteries extend, such as the renal arteries. Patients diagnosed with aortic aneurysms near or involving the renal arteries are presently considered poor candidates for endovascular grafting as currently available endovascular grafting systems are often not suitable for use in this region. Currently available endovascular grafts typically require a region of at least one (1) to one and a half (1.5) centimeters of non-aneurysmic aorta 28 proximal to the aneurysm to provide a region where the end of the graft may be securely anchored in place. Deployment of endovascular grafts within branched regions of the aorta such as near the renal or subclavian arteries presents additional challenges for the graft to be implanted without blocking or restricting blood flow into the branch arteries.

There remains a need in the art for new endovascular grafting systems and methods that are usable for endovascular grafting of aneurysms in regions of a blood vessel from which branch blood vessels extend.

### BRIEF DESCRIPTION OF THE DRAWINGS

FIG. 1 is a schematic of a human body.

FIG. 2 is a view of an endoluminal graft implanted in an aneurysmic portion of the abdominal aorta according to the prior art.

FIG. 3 is a view of an endoluminal graft implanted in an aneurysmic portion of the abdominal aorta according to one embodiment of the present invention.

FIG. 4 is a partial cross sectional view of FIG. 3 taken along line 4-4.

FIG. 5 is a view of an endoluminal graft implanted in an aneurysmic portion of the abdominal aorta according to another embodiment of the present invention.

FIG. 6 is a view of an endoluminal graft implanted in an aneurysmic portion of the abdominal aorta according to yet another embodiment of the present invention.

FIG. 7A is a side perspective view of still another embodiment of the present invention.

FIG. 7B is a side perspective view of the embodiment of the present invention shown in FIG. 7A.

FIG. 8A is a top plan view of another embodiment of the present invention.

FIG. 8B is a top plan view of the embodiment of the present invention shown in FIG. 8A.

FIG. 9 is a top plan view of yet another embodiment of the present invention.

FIG. 10 is a perspective view of the embodiment of the present invention shown in FIG. 9.

FIG. 11 is a side view of still another embodiment of the present invention.

FIG. 12 is a perspective view of another embodiment of the present invention.

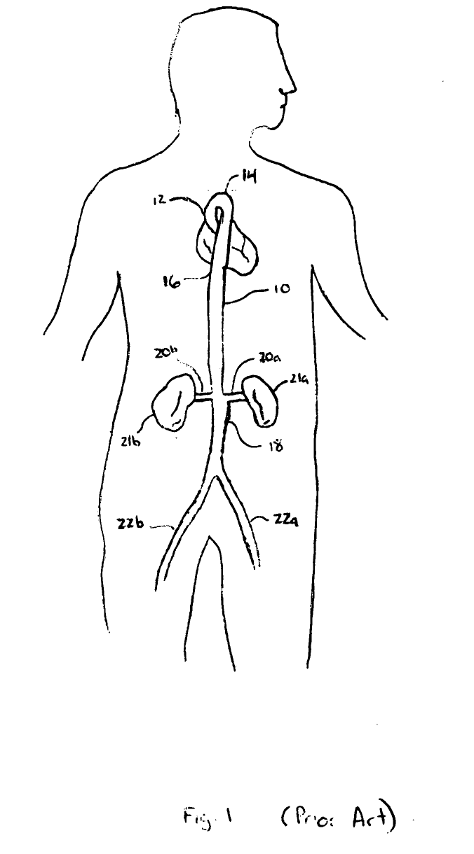

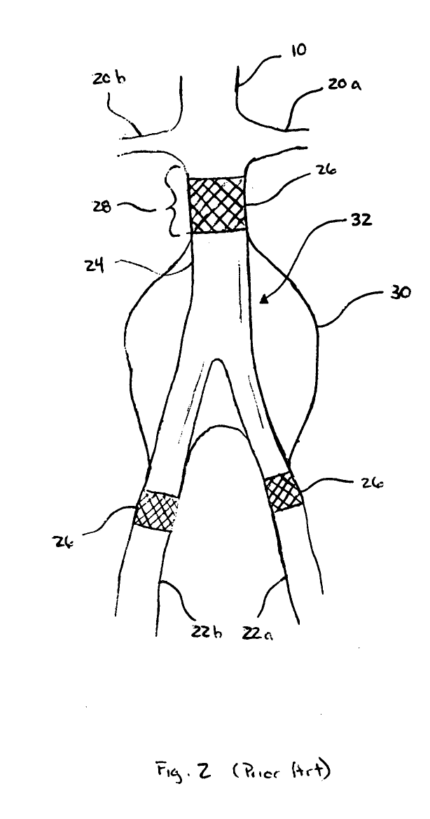

### DESCRIPTION OF THE ILLUSTRATED EMBODIMENTS

For the purposes of promoting understanding of the principles of the invention, reference will now be made to the embodiments illustrated in the drawings and specific language will be used to describe the same. It will nevertheless be understood that no limitation of the scope of the invention is hereby intended and alterations and modifications in the illustrated device, and further applications of the principles of the present invention as illustrated herein being contemplated as would normally occur to one skilled in the art to which the invention relates.

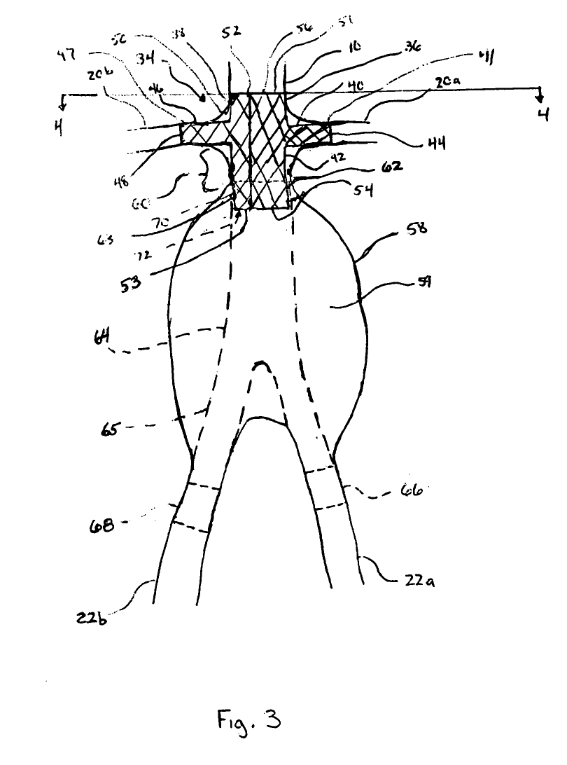

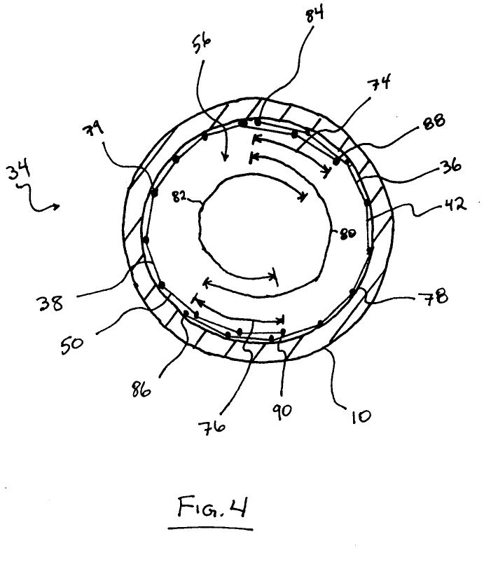

FIG. 3 is a schematic view of a branch vessel stent graft 34 according to one embodiment of the present invention. The illustrated placement of stent graft 34 is at the junction between aorta 10 and renal arteries 20a and 20b. Other embodiments of stent graft 34 are placed at other junctions between two or more anatomical vessels. In the illustrated embodiment, branch vessel stent graft 34 includes a first stent portion 36 engaged with a second stent portion 38 which define a lumen 56, as also shown in FIG. 4.

Stent portion 36 includes a proximal end 51, a distal end 54, a stent wall 42 and a lateral stent 40 extending from stent wall 42 and having a lumen 44 therethrough. Stent wall 42 is generally shaped as an open-cylinder when placed in aorta 10 and substantially contacts the inside wall of aorta 10. In other embodiments, the contact between stent wall 42 and the inside wall of aorta 10 is less than substantial. Prior to placement in aorta 10, stent wall 42 can be relatively flat or curved. Stent wall 42 comprises a lattice, or mesh, type construction using a form-returning material with at least one aperture. Alternate embodiments utilize shape memory alloys, such as Nitinol. Other embodiments comprise different construction, such as solid surfaces, while still other embodiments comprise lattice construction with no apertures. The aperture in stent wall 42 is sized to allow the passage of blood, or other body fluid, through stent wall 42.

An open-cylinder is a surface traced by a straight line moving parallel to a fixed straight line and intersecting a fixed planar open curve, where the length of the straight line may vary during the moving. It is understood that the term “open curve” includes a line, implying that a flat plane is one embodiment of an open-cylinder. Alternately, a closed-cylinder is a surface traced by a straight line moving parallel to a fixed straight line and intersecting a fixed planar closed curve, where the length of the straight line may vary during the moving. Furthermore, a cylindrical object is an object resembling either an open-cylinder or a closed-cylinder.

Lateral stent 40 is generally shaped as a closed-cylinder. In other embodiments, lateral stent 40 is generally shaped as an open-cylinder. Lateral stent 40 is off-center relative to stent wall 42, that is, nearer to proximal end 51 than to distal end 54, and includes an anchor portion 41. The off-center arrangement results in placement of a longer portion of stent wall 42 inferior to, or below, renal artery 20a. The off-center arrangement can further increase the versatility of stent portion 36 since stent portion 36 may be inverted if a longer portion of stent wall 42 is required superior to, or above, renal artery 20a. In other embodiments, lateral stent 40 is equidistant from proximal end 51 and distal end 54. Lateral stent 40 may be attached to stent portion 36 at various locations to accommodate different anatomical vessel structures. Distal end 54 includes a sealing portion 62 which will be described in greater detail further below.

Stent portion 38 includes a proximal end 52, a distal end 53, a stent wall 50 and a lateral stent 46 extending from stent wall 50 and having a lumen 48 therethrough. Stent wall 50 is generally shaped as an open-cylinder when placed in aorta 10 and substantially contacts the inside wall of aorta 10. In other embodiments, the contact between stent wall 50 and the inside wall of aorta 10 is less than substantial. Prior to placement in aorta 10, stent wall 50 can be relatively flat or curved. Stent wall 50 comprises a lattice, or mesh, type construction with at least one aperture. Other embodiments comprise different construction, such as solid surfaces, while still other embodiments comprise lattice construction with no apertures. The aperture in stent wall 50 is sized to allow the passage of blood, or other body fluid, through stent wall 50.

Lateral stent 46 is generally shaped as a closed-cylinder. In other embodiments, lateral stent 46 is generally shaped as an open-cylinder. Lateral stent 46 is off-center relative to stent wall 50, that is, nearer to proximal end 52 than to distal end 53, and includes an anchor portion 47. The off-center arrangement results in placement of a longer portion of stent wall 50 inferior to, or below, renal artery 20b. The off-center arrangement can further increase the versatility of stent portion 38 since stent portion 38 may be inverted if a longer portion of stent wall 50 is required superior to, or above, renal artery 20b. In other embodiments, lateral stent 46 is equidistant from proximal end 52 and distal end 53. Distal end 53 includes a sealing portion 62 which will be described in greater detail further below.

FIG. 4 is a partial cross-sectional view of branch graft 34 as shown in FIG. 3 taken along line 4-4. In this particular embodiment, stent portion 36 is anchored to aorta 10 at a plurality of contact points 78. In alternate embodiments, stent portion 36 is anchored to aorta 10 using other suitable anchoring means such as hooks, barbs, rings and/or clips. Similarly, stent portion 38 is anchored to aorta 10 at a plurality of contact points 79. In alternate embodiments, stent portion 38 is anchored to aorta 10 using other suitable anchoring means such as hooks, barbs, rings and/or clips. As shown in FIG. 4, stent wall 42 includes a first end 84 and a second end 86 and has an arc length 80. Arc length 80 is approximately two-thirds (⅔) or more of the inner circumference of aorta 10. In alternative embodiments, arc length 80 is less than two-thirds (⅔) of the inner circumference of aorta 10. In still other embodiments, arc length 80 is greater than the inner circumference of aorta 10, thereby allowing stent portion 36 to individually cover at least one circumferential ring of the inner surface of aorta 10, where a circumferential ring is any closed loop that defines a minimum distance around a closed-cylinder.

Stent wall 50 includes a first end 88 and a second end 90 and has an arc length 82. Arc length 82 is approximately two-thirds (⅔) or more of the inner circumference of aorta 10. In alternative embodiments, arc length 82 is less than two-thirds (⅔) of the inner circumference of aorta 10. Optionally, arc length 80 is approximately equal to, less than or greater than arc length 82. The sum of arc length 80 and arc length 82 is approximately equal to at least the inner circumference of aorta 10, thereby allowing stent wall 42 and stent. wall 50 to combine to cover at least one circumferential ring of the inner surface of aorta 10, as depicted in FIG. 4. In other embodiments, the sum of arc length 80 and arc length 82 is approximately equal to at most the inner circumference of aorta 10.

When stent portion 38 is expanded after stent portion 36 is implanted as previously described, a portion of stent wall 50 is deployed within and engages stent wall 42 as shown in FIG. 4. A first overlap portion 74 is located between end 84 and end 88. A second overlap portion 76 is located between end 86 and end 90. The combined arc lengths of first overlap portion 74 and second overlap portion 76 are equal to approximately one-third (⅓) or more of the inner circumference of aorta 10. Optionally, first overlap portion 74 and second overlap portion 76 include a sealing means such as hooks, pins, adhesives and the like. In alternative embodiments of the present invention, the combined arc lengths of first overlap portion 74 and second overlap portion 76 are less than one-third (⅓) of the inner circumference of aorta 10. In still other embodiments, the combined arc lengths of first overlap portion 74 and second overlap portion 76 are more than one-third (⅓) of the inner circumference of aorta 10.

In one embodiment, stent portion 36 and stent portion 38 are self-expanding and fabricated from a suitable form-returning material. In other embodiments, stent portion 36 and stent portion 38 are pressure expandable and fabricated from a suitable material such as wire or some other plastically deformable material.

Treatment of aneurysm 58 using stent graft 34 and a bifurcated endovascular graft 64 having a fluid-tight sheath 65 will now be described. Shown in FIG. 3 is a subrenal abdominal aortic aneurysm 58 with iliac involvement. In this particular example, less than one and one half (1.5) centimeters of non-aneurysmic aorta 60 is proximal to aneurysm 58. The proximity of aneurysm 58 to renal arteries 20a and 20b renders treatment using traditional endovascular grafts undesirable. Endovascular graft 64 having a proximal end 70, a first distal end 66, a second distal end 68 and a lumen 72 therethrough is deployed in the aneurysmic portion 58 of aorta 10. In this particular example, distal branches 66 and 68 are deployed and secured in iliac arteries 22a and 22b, respectively. Proximal end 70 is deployed and secured to non-aneurysmic aorta 60 proximal to aneurysm 58. Endovascular graft 64 may be self-expanding or pressure expanding as is known in the art. Endovascular graft 64 is deployed using radiographic visualization to ensure precise alignment within the vascular lumen, although other embodiments utilize other methods of alignment.

Once endovascular graft 64 is deployed, stent graft 34 is deployed. In this particular example, stent graft 34 is self-expanding, although in other embodiments, the stent graft is pressure-expanding. First, stent portion 36 is inserted in a collapsed state via a body introduction device into a suitable artery (e.g., the femoral artery) and advanced using radiographic guidance to the intended implantation site. Stent portion 36 is deployed over a guide wire. Lateral stent 40 is oriented so that its open end is disposed in a cephalad position along proximal end 51 when stent portion 36 is in the collapsed state. In other embodiments, lateral stent 40 is disposed in alternate positions when stent portion 36 is in the collapsed state. For example, lateral stent 40 may be oriented so that its open end is disposed in a caudal position so that its open end is along the distal end 54, or it may be axially collapsed upon itself such that its open end is disposed along neither the proximal end 51 nor the distal end 54. Lateral stent 40 is guided into renal artery 20a and distal end 54 is inserted into lumen 72 of graft 64.

Once properly positioned, stent portion 36 is expanded such that stent portion 36 is anchored within aorta 10. In the illustrated embodiment, stent portion 36 is expanded in a primarily radial direction, although other embodiments primarily expand in other directions. In the event stent portion 36 is expanded while not located at, but sufficiently near, the proper position, the pressure that stent portion 36 exerts on aorta 10 and renal artery 20a will result in stent portion 36 automatically aligning, or self-aligning, itself in the proper position. Other embodiments utilize stents that self-align toward the proper position, while still other embodiments do not automatically center toward the proper position. In the example embodiment, lateral stent 40 becomes anchored to renal artery 20a and stent wall 42 becomes anchored to aorta 10 inferior and superior to renal artery 20a. Lumen 44 ensures proper blood flow through renal artery 20a. The anchoring is accomplished by the pressure exerted by stent portion 36 on aorta 10 and renal artery 20a, although other embodiments utilize other means of anchoring, such as hooks, barbs, clips and/or sutures, by way of nonlimiting example.

As stent portion 36 expands, sealing portion 62 of stent portion 36 is brought into sealable contact with proximal end 70 of endovascular graft 64. The seal between proximal end 70 and sealing portion 62 may be mechanical (e.g., hooks, pins and/or the like), frictional, chemical (e.g., adhesive or fusing agent) or any combination of the three. The seal between proximal end 70 and sealing portion 62 may be fluid-tight to prevent leakage through the seal and into the aneurysmal vessel 59, although other embodiments utilize a seal that is not fluid-tight.

Next, stent portion 38 is inserted in a collapsed state via a body introduction device into a suitable artery (e.g., the femoral artery) and advanced using radiographic guidance to the intended implantation site. Stent portion 38 is deployed over a guide wire. Lateral stent 46 is disposed with its open end in a cephalad position along proximal end 52 when stent portion 38 is in the collapsed state. In other embodiments, lateral stent 46 may be disposed relative to stent portion 38 as discussed above with respect to lateral stent 40 and stent portion 36. Lateral stent 46 is guided into renal artery 20b and distal end 53 is inserted into lumen 72 of graft 64.

Once properly positioned, stent portion 38 is expanded such that stent portion 38 is anchored within aorta 10. In the illustrated embodiment, stent portion 38 is expanded in a primarily radial direction, although other embodiments primarily expand in other directions. In the event stent portion 38 is expanded while not located at, but sufficiently near, the proper position, the pressure that stent portion 38 exerts on aorta 10 and renal artery 20b will result in stent portion 38 automatically aligning, or self-aligning, itself in the proper position. Other embodiments utilize stents that self-align toward the proper position, while still other embodiments do not automatically center toward the proper position. In the example embodiment, lateral stent 46 becomes anchored to renal artery 20b and stent wall 56 becomes anchored to aorta 10 inferior and superior to renal artery 20b. Lumen 48 ensures proper blood flow through renal artery 20b. The anchoring is accomplished by the pressure exerted by stent portion 38 on aorta 10 and renal artery 20b, although other embodiments utilize other means of anchoring, such as hooks, barbs, clips and/or sutures, by way of nonlimiting example.

As stent portion 38 expands, sealing portion 63 of stent portion 38 is brought into sealable contact with proximal end 70 of graft 64. The seal between proximal end 70 and sealing portion 63 may be mechanical (e.g., hooks, pins and/or the like), frictional, chemical (e.g., adhesive or fusing agent) or any combination of the three. The seal between proximal end 70 and sealing portion 63 is fluid-tight so as to prevent leakage through the seal and into the aneurysmal vessel 59, although other embodiments may have a seal between proximal end 70 and sealing portion 63 that is not fluid-tight.

Once stent portion 36 and stent portion 38 are deployed and engaged as shown in FIGS. 3 and 4, blood flowing through lumen 56 passes through branch graft 34 and into endovascular graft 64. Sealing portions 62 and 63 prevent leakage of blood between branch graft 34 and endovascular graft 64 and into aneurysmal vessel 59. Lateral stent 40 and lateral stent 46 ensure proper blood flow through renal arteries 20a and 20b, respectively.

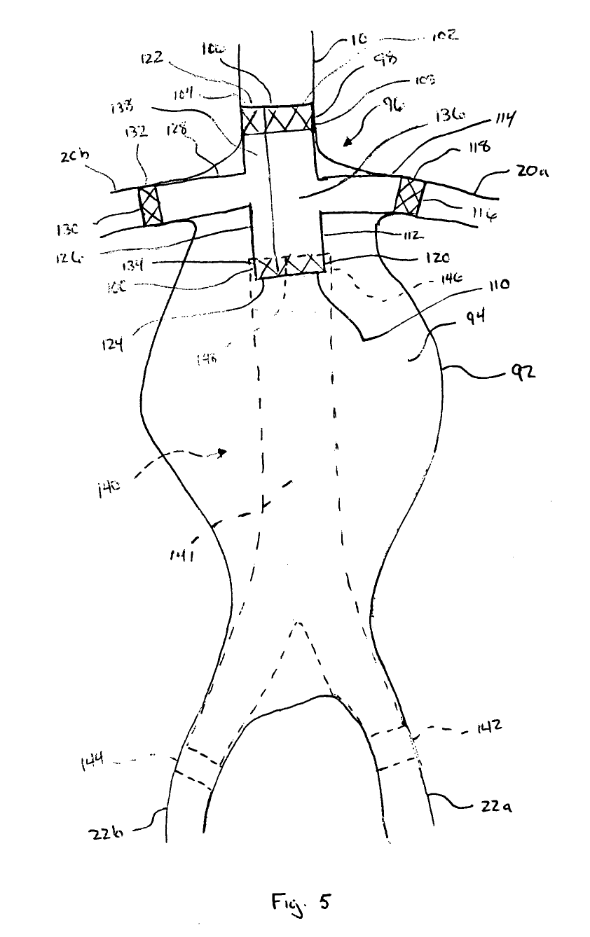

A subrenal abdominal aortic aneurysm 92 with iliac and renal involvement and a branch vessel stent graft 96 according to an another embodiment are shown in FIG. 5. In this particular example, no non-aneurysmic aorta inferior to renal arteries 20a and 20b is proximal to aneurysm 92. The involvement of renal arteries 20a and 20b with aneurysm 92 renders treatment using traditional endovascular grafts undesirable.

Branch vessel stent graft 96 includes a first stent portion 102 engaged with a second stent portion 104 which define a lumen 106. The illustrated placement of stent graft 96 is at the junction between aorta 10 and renal arteries 20a and 20b. Other embodiments of stent graft 96 are placed at other junctions between anatomical vessels. Stent portion 102 includes an anchor portion 98, a proximal end 108, a distal end 110, a stent wall 112 and a lateral stent 114 extending from stent wall 112 and having a lumen 116 therethrough. Stent wall 112 is generally shaped as an open-cylinder when placed in aorta 10 and substantially contacts the inside wall of aorta 10. In other embodiments, the contact between stent wall 112 and the inside wall of aorta 10 is less than substantial. Prior to placement in aorta 10, stent wall 112 can be relatively flat or curved.

Lateral stent 114 is generally shaped as an closed-cylinder. In other embodiments, lateral stent 114 is generally shaped as an open-cylinder. Lateral stent 114 is off-center relative to stent wall 112, that is, nearer to proximal end 108 than to distal end 110, and includes an anchor portion 118. The off-center arrangement results in placement of a longer portion of stent wall 112 inferior to, or below, renal artery 20a. The off-center arrangement can further increase the versatility of stent portion 102 since stent portion 102 may be inverted if a longer portion of stent wall 112 is required superior to, or above, renal artery 20a. In other embodiments, lateral stent 114 is equidistant from proximal end 108 and distal end 110. Distal end 110 includes a sealing portion 120 similar to sealing portions 62 and 63 described previously.

Stent portion 102 further includes a cover 136, which comprises a pliable material such as expanded polytetrafluoroethylene (ePTFE), woven polyester or other suitable, fluid-tight covering material. In the embodiment shown in FIG. 5, outer portions of stent wall 112 and lateral stent 114 are covered by fluid-tight material. In alternative embodiments (not shown), a greater or lesser portion of stent portion 102 is covered by fluid-tight material. In yet another embodiment, all of stent portion 102 is covered by fluid-tight material. In still other embodiments, inner portions of stent portion 102 are covered by fluid-tight material.

Second stent portion 104 includes a proximal end 122, a distal end 124, a stent wall 126 and a lateral stent 128 extending from stent wall 126 and having a lumen 130 therethrough. Stent wall 126 is generally shaped as an open-cylinder when placed in aorta 10 and substantially contacts the inside wall of aorta 10. In other embodiments, the contact between stent wall 126 and the inside wall of aorta 10 is less than substantial. Prior to placement in aorta 10, stent wall 126 can be relatively flat or curved.

Lateral stent 128 is generally shaped as an closed-cylinder. In other embodiments, lateral stent 128 is generally shaped as an open-cylinder. Lateral stent 128 is off-center relative to stent wall 126, that is, nearer to proximal end 122 than to distal end 124, and includes an anchor portion 132. The off-center arrangement results in placement of a longer portion of stent wall 126 inferior to, or below, renal artery 20b. The off-center arrangement can further increase the versatility of stent portion 104 since stent portion 104 may be inverted if a longer portion of stent wall 126 is required superior to, or above, renal artery 20b. In other embodiments, lateral stent 128 is equidistant from proximal end 122 and distal end 124. Distal end 124 includes a sealing portion 134 similar to sealing portions 62 and 63 described previously. Stent portion 104 further includes a cover 138, which comprises a pliable material such as ePTFE, woven polyester or other suitable, fluid-tight covering material. In the embodiment shown in FIG. 5, portions of stent wall 126 and lateral stent 128 are covered by fluid-tight material. In alternative embodiments (not shown), a greater or lesser portion of stent portion 104 is covered by fluid-tight material. In yet another embodiment, all of stent portion 104 is covered by fluid-tight material. In still other embodiments, a fluid-tight material lines at least a portion of the inside of stent portion 104.

In one embodiment of the present invention, stent portion 102 and stent portion 104 are self-expanding and fabricated from a suitable form-returning material. In other embodiments, stent portion 102 and stent portion 104 are pressure expandable and fabricated from a suitable material such as wire or some other plastically deformable material.

Deployment of stent graft 96 and a bifurcated endovascular graft 140 having a fluid-tight sheath 141 is similar to deployment of stent graft 34 and endovascular graft 64 as previously described with respect to FIGS. 3 and 4. Endovascular graft 140 includes distal branches 142 and 144, proximal end 146 and lumen 148. Distal branches 142 and 144 are deployed and secured in iliac arteries 22a and 22b, respectively.

Once endovascular graft 140 is deployed, stent graft 96 is deployed. In this particular example, stent graft 96 is self-expanding, although in other embodiments, the branch graft is pressure-expanding. First, stent portion 102 is inserted in a collapsed state via a body introduction device into a suitable artery (e.g., the femoral artery) and advanced using radiographic guidance to the intended implantation site. Stent portion 102 is deployed over a guide wire. Lateral stent 114 is disposed so that it extends cephaladly along proximal end 108 when stent portion 102 is in the collapsed state. In other embodiments, lateral stent 114 is disposed in alternate positions when stent portion 102 is in the collapsed state. For example, lateral stent 114 may extend caudally along distal end 110, or it may be axially collapsed upon itself such that it is disposed along neither the proximal end 108 nor the distal end 110. Lateral stent 114 is guided into renal artery 20a and distal end 110 is inserted into lumen 148 of graft 140.

Once properly positioned, stent portion 102 is expanded such that stent portion 102 is anchored within aorta 10, lateral stent 114 becomes anchored to renal artery 20a and stent wall 112 becomes anchored to aorta 10 superior to renal artery 20a. In the illustrated embodiment, stent portion 102 is expanded in a primarily radial direction, although other embodiments primarily expand in other directions. In the event stent portion 102 is expanded while not located at, but sufficiently near, the proper position, the pressure that stent portion 102 exerts on aorta 10 and renal artery 20a will result in stent portion 102 automatically aligning, or self-aligning, itself in the proper position. Other embodiments utilize stents that self-align toward the proper position, while still other embodiments do not automatically center toward the proper position.

As stent portion 102 expands, sealing portion 120 of stent portion 102 is brought into sealable contact with proximal end 146 of endovascular graft 140. The seal between proximal end 146 and sealing portion 120 may be mechanical (e.g., hooks, pins and/or the like), frictional, chemical (e.g., adhesive or fusing agent) or any combination of the three. The seal between proximal end 146 and sealing portion 120 is fluid-tight so as to prevent leakage through the seal and into the aneurysmal vessel 94, although other embodiments may have a seal between proximal end 146 and sealing portion 120 that is not fluid-tight.

Next, stent portion 104 is inserted in a collapsed state via a body introduction device into a suitable artery (e.g., the femoral artery) and advanced using radiographic guidance to the intended implantation site. Stent portion 104 is deployed over a guide wire. Lateral stent 128 is disposed so that it extends cephaladly along proximal end 122 when stent portion 104 is in the collapsed state. In other embodiments, lateral stent 128 may be disposed in alternate positions as discussed above with respect to stent portion 102 and lateral stent 114. Lateral stent 128 is guided into renal artery 20b and distal end 124 is inserted into lumen 148 of graft 140.

Once properly positioned, stent portion 104 is expanded such that stent portion 104 is anchored within aorta 10, lateral stent 128 becomes anchored to renal artery 20b and stent wall 126 becomes anchored to aorta 10 superior to renal artery 20b. In the illustrated embodiment, stent portion 104 is expanded in a primarily radial direction, although other embodiments primarily expand in other directions. In the event stent portion 104 is expanded while not located at, but sufficiently near, the proper position, the pressure that stent portion 104 exerts on aorta 10 and renal artery 20b will result in stent portion 104 automatically aligning, or self-aligning, itself in the proper position. Other embodiments utilize stents that self-align toward the proper position, while still other embodiments do not automatically center toward the proper position.

As stent portion 104 expands, sealing portion 134 of stent portion 104 is brought into sealable contact with proximal end 146 of graft 140. The seal between proximal end 146 and sealing portion 134 may be mechanical (e.g., hooks, pins and/or the like), frictional, chemical (e.g., adhesive or fusing agent) or any combination of the three. The seal between proximal end 146 and sealing portion 134 is fluid-tight so as to prevent leakage through the seal and into the aneurysmal vessel 94, although other embodiments may have a seal between proximal end 146 and sealing portion 134 that is not fluid-tight.

Once stent portion 102 and stent portion 104 are deployed and engaged as shown in FIG. 5, blood flowing through lumen 106 passes through stent graft 96 and into endovascular graft 140. Sealing portions 120 and 134 prevent leakage of blood between branch graft 96 and endovascular graft 140 and into aneurysmal vessel 94. Coverings 136 and 138 prevent leakage of blood from branch graft 96 into aneurysmal vessel 94. Lateral stent 114 and lateral stent 128 ensure proper blood flow through renal arteries 20a and 20b, respectively.

Frequently, aneurysms occur in areas where there are three or more branching blood vessels, such as where three or more renal arteries branch from the aorta. In these situations, a stent graft comprising three or more stent portions may be used to collectively repair the aneurysm. The arc-lengths of each stent portion and the size of each lateral stent can be adjusted to accommodate a variety of different vessel sizes. Additionally, the stent portions may be individually adjusted along the axial length of the vessel to accommodate variations in the axial locations of various branch vessels.

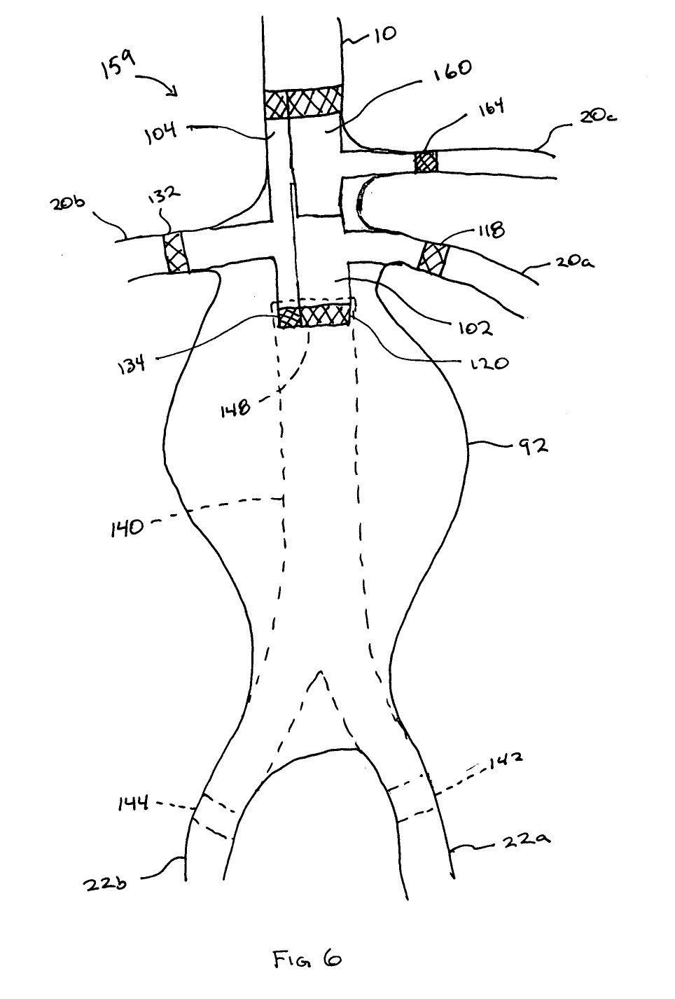

An embodiment of the present invention used to repair a subrenal abdominal aortic aneurysm affecting three renal arteries is depicted in FIG. 6. This example embodiment is similar to the embodiment depicted in FIG. 5, except as otherwise stated. In this particular example, renal arteries 20a, 20b and 20c are affected by aneurysm 92 rendering treatment using traditional endovascular grafts undesirable.

Branch vessel stent graft 159 includes first stent portion 102, second stent portion 104 and third stent portion 160. Stent portion 102 engages stent portions 104 and 160, while stent portion 104 further engages stent portion 160. Other embodiments utilize different arrangements of stent portions to coincide with the particular arrangement of branch vessels. Stent portion 160 includes anchor portion 164, which is positioned inside renal artery 20c.

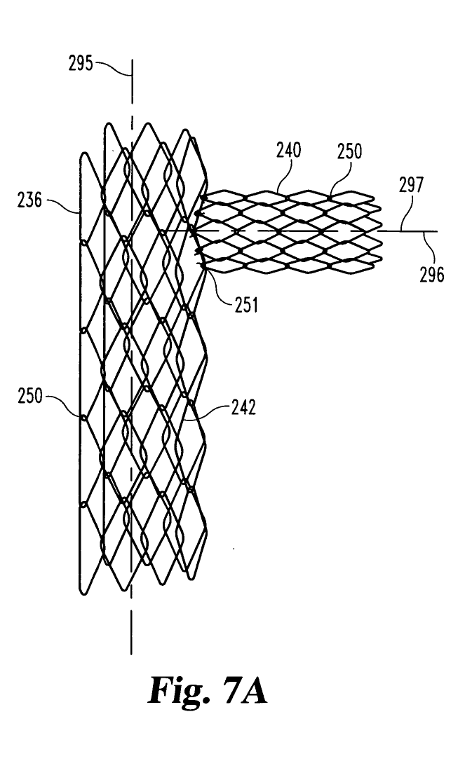

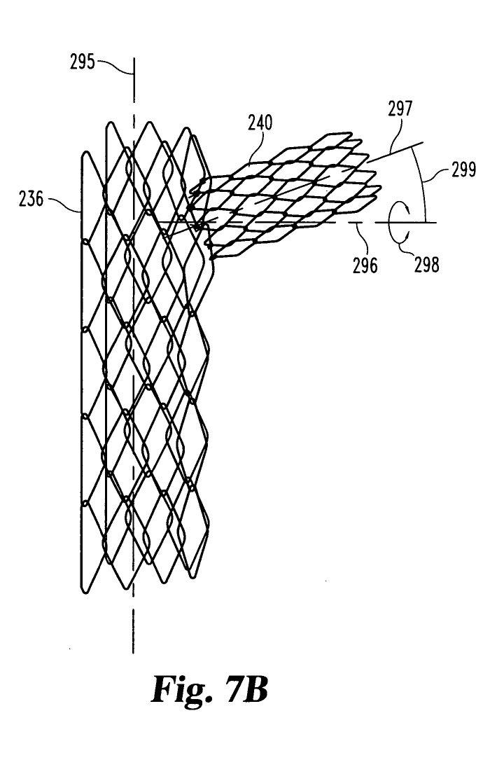

In other embodiments, the first and second lateral stents are angularly disposed relative to one another and/or relative to their respective stent portions to accommodate anatomical vessels that differ in the size, location and/or orientation. An example branch vessel stent graft having lateral stents disposed at an angle relative to stent portion walls is shown in FIGS. 7A and 7B. Stent portion 236 defines stent portion reference axis 295, and lateral stent 240 defines lateral stent axis 297. Lateral stent reference axis 296 reflects the orientation of lateral stent 240 in a non-deflected state, and is coincident with lateral stent axis 297 when lateral stent 240 is not deflected. In this example, lateral stent reference axis 296 is orthogonal to stent portion reference axis 295. In other embodiments, reference axis 296 is non-orthogonally angled with respect to reference axis 295.

In still other embodiments, the connection between the lateral stent and the open-cylinder stent portion allows the lateral stent to move in relation to the open-cylinder stent portion. In certain embodiments, the lateral stent is movable up to forty degrees (40°) in any direction from the lateral stent's reference location—the lateral stent can be moveable to circumscribe an eighty degree (80°) cone.

Stent portion 236 and lateral stent 240 each comprise a lattice, or mesh, construction using a form-returning material with a plurality of apertures. The individual components of each lattice are connected at connection locations 250. The connection locations utilize an interlinking structure between the individual lattice components to form stent wall 242. Alternate embodiments utilize additional flexible binding material, for example a suture, that is tied to connect the individual components of the lattice together. Still other embodiments utilize additional bendable binding material, for example a bendable metal, that is twisted to connect the individual components of the lattice together. The connection locations 251 between lateral stent 240 and stent portion 236 are similar to connection locations 250.

FIG. 7B depicts lateral stent 240 in a deflected orientation with lateral stent axis 297 angularly displaced by a lateral stent displacement angle 299 from lateral stent reference axis 296. In the example embodiment, displacement angle 299 is at most forty degrees (40°). In other embodiments, lateral stent displacement angle 299 is less than ninety degrees (90°). Lateral stent axis 297, and consequently lateral stent 240, may be angularly displaced in any direction within a cone described by rotating lateral stent axis 297 around lateral stent reference axis 296 when displacement angle 299 is equal to the maximum deflection angle, as indicated by rotation arrow 298. The size of the cone inside which lateral stent axis 297 may be positioned is defined by a value that is twice the maximum lateral stent displacement angle 299, for example, a maximum displacement angle 299 equal to sixty degrees (60°) describes a one hundred twenty degree (120°) cone in which lateral stent axis 297 may be positioned.

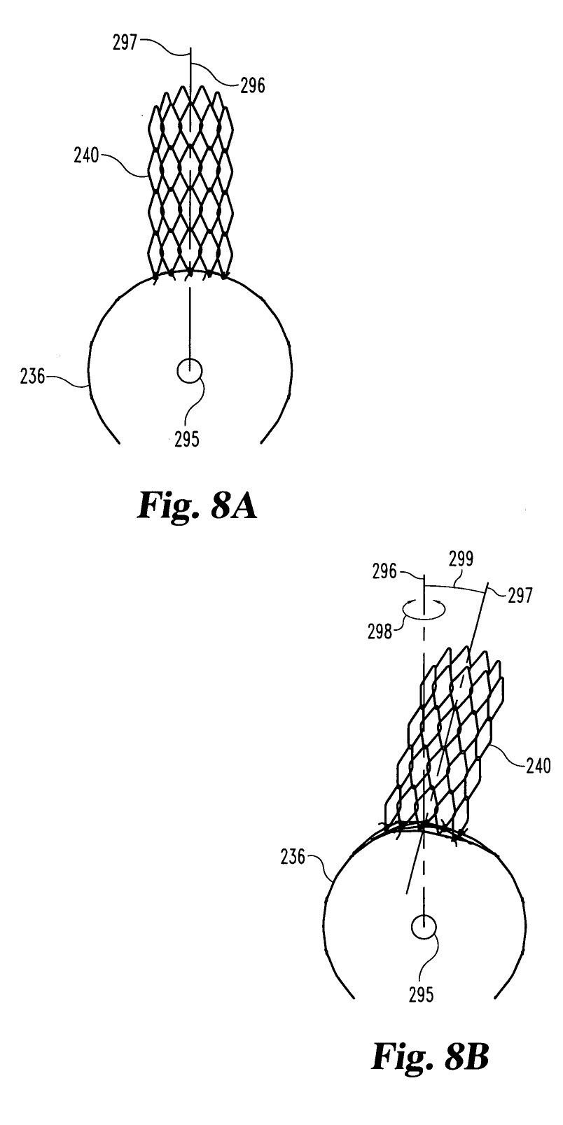

FIGS. 8A and 8B depict lateral stent deflection in a direction different than that depicted in FIGS. 7A and 7B. FIG. 8A depicts lateral stent 240 in a non-deflected orientation. FIG. 8B depicts lateral stent 240 deflected by lateral stent displacement angle 299 in a direction approximately perpendicular to the deflection direction depicted in FIG. 7B.

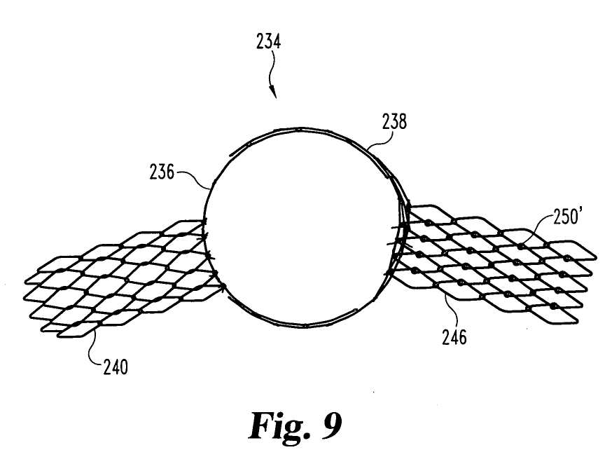

FIG. 9 depicts branch stent graft 234, which includes second stent portion 238 partially overlapping first stent portion 236. Lateral stent 240 is attached to stent portion 236, and lateral stent 246 is attached to stent portion 238. In this example embodiment, lateral stent 246 is angled while lateral stent 240 is not. Additionally, the individual lattice components of lateral stent 246 are connected at connection locations 250′, which depict a looping overlap type connection. Alternate embodiments utilize multiple looping overlap construction for connection locations 250′.

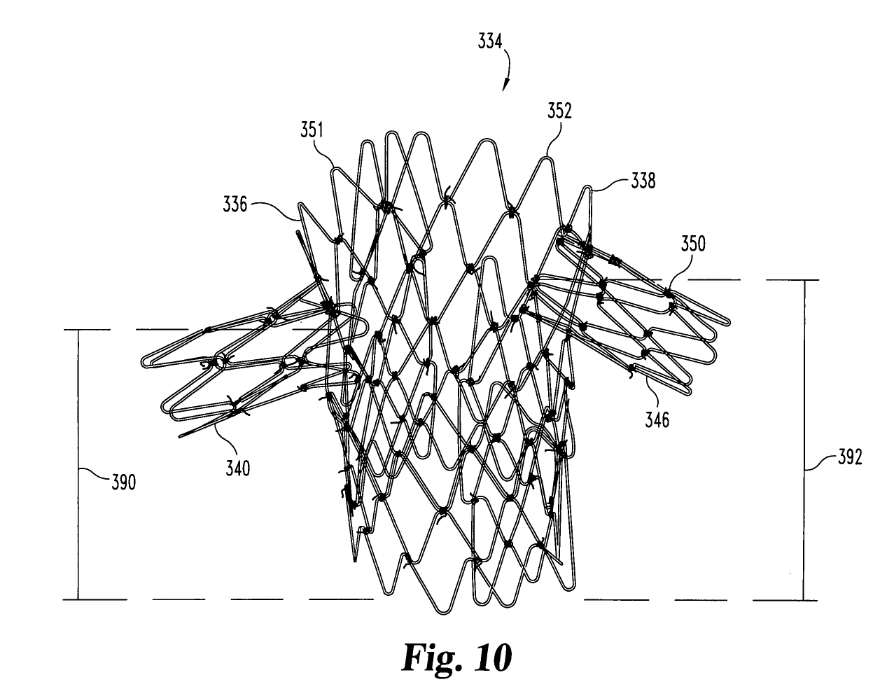

FIG. 10 depicts stent graft 334. In this particular example, lateral stent 340 is attached to stent portion 336 at a location displaced a distance 390 from distal end 354, and lateral stent 346 is attached to stent portion 338 at a location displaced a distance 392 from distal end 353, where distance 392 is greater than distance 390. Connection locations 350 utilize sutures to connect the individual components of stent portions 336 and 338 together, the individual components of lateral stents 340 and 346 together, lateral stent 340 to stent portion 336, and lateral stent 346 to stent portion 338. Additionally, distal end 354 is aligned with distal end 353, and proximal end 351 is aligned with proximal end 352. In other embodiments, distal end 354 is offset from distal end 353, and in still other embodiments, proximal end 351 is offset from proximal end 352.

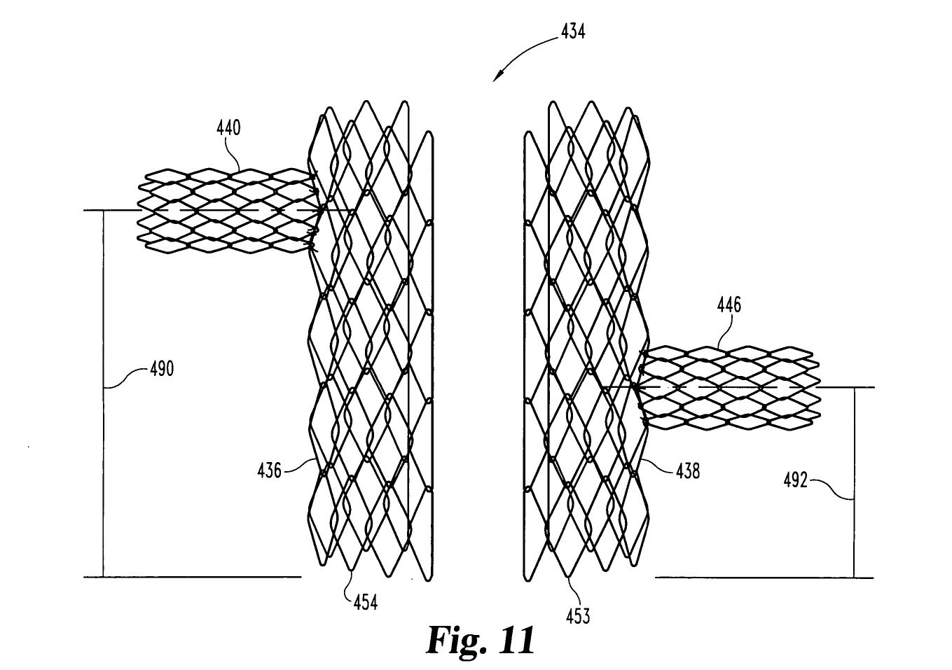

FIG. 11 depicts another embodiment branch stent graft 434 in a partially exploded view. Lateral stent 440 is attached to stent portion 436 at a location that is a distance 490 from distal end 454. Lateral stent 446 is attached to stent portion 438 at a location that is a distance 492 from distal end 453. In this illustrated embodiment, distance 490 is equal to distance 492.

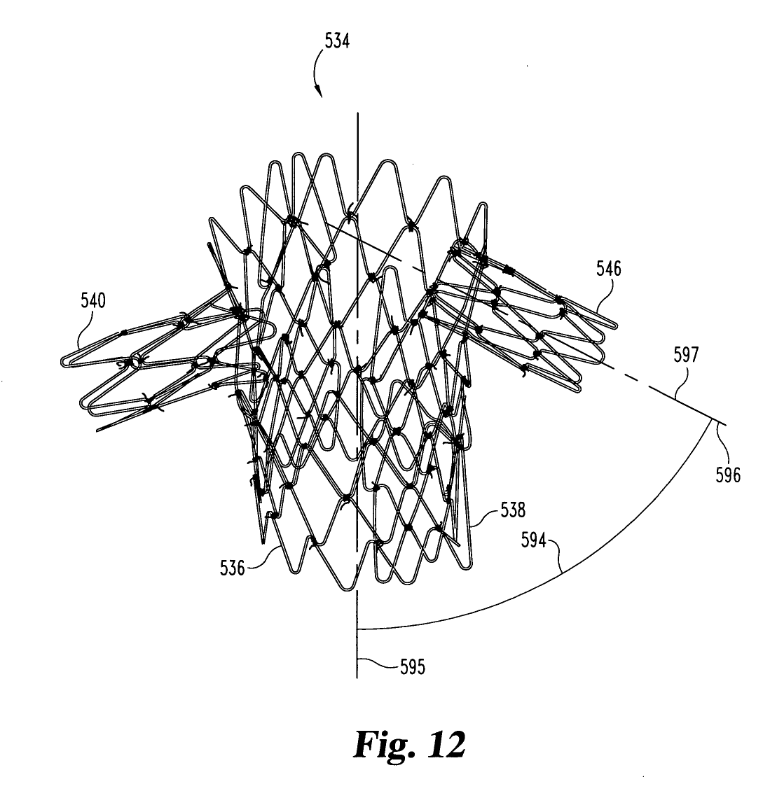

FIG. 12 depicts yet another example embodiment branch stent graft 534. Lateral stent portion 540 is attached to stent portion 536, and lateral stent portion 546 is attached to stent portion 538. In this illustrated embodiment, the non-deflected orientation of lateral stent 546 is offset by angle 594 from stent portion reference axis 595. Since lateral stent 546 is in a non-deflected orientation, lateral stent axis 597 is coincident with a lateral stent reference axis 596. Lateral stent 546 may be deflected from this reference orientation by up to approximately forty degrees (40°). In other embodiments, lateral stent 546 may be deflected from its reference orientations by angles exceeding forty degrees (40°).

While the invention has been illustrated and described in detail in the drawings and foregoing description, the same is to be considered as illustrative and not restrictive in character, it being understood that only example embodiments have been shown and described and that all changes, modifications and equivalents that come within the spirit of the inventions disclosed are desired to be protected. The articles “a”, “an”, “said” and “the” are not limited to a singular element, and include one or more such elements.

1. A frame for an anatomical vessel with an inner surface defining an inner circumference and at least one connecting branch vessel with an inner surface defining an inner circumference, comprising:
a first open-cylinder member with an outer surface defining a circumferential arc length, first and second opposing end portions, and a connection location, wherein
said circumferential arc length is at most equal to the anatomical vessel inner circumference, wherein
said first open-cylinder member is adapted for placement within the lumen of the anatomical vessel with a substantial portion of said first open-cylinder member outer surface contacting the anatomical vessel inner surface, wherein
said connection location is nearer to said first opposing end than said second opposing end; and
a first cylindrical connecting member connected to said first open-cylinder member at said connection location, said first cylindrical connecting member adapted for placement within the lumen of the first connecting branch vessel with a substantial portion of said first cylindrical connecting member outer surface contacting at least one branch vessel inner surface.

2. The frame of claim 1, wherein said first open-cylinder member is adapted to exert pressure on the anatomical vessel inner surface and said first cylindrical connecting member is adapted to exert pressure on the first connecting branch vessel inner surface, wherein the exerted pressures act to center said first open-cylinder member and said first cylindrical connecting member in the junction between the anatomical vessel and the at least one connecting branch vessel.

3. The frame of claim 1, wherein
said first open-cylinder member further comprises at least one aperture through said first open-cylinder member outer surface;
said first cylindrical connecting member further comprises at least one aperture through said first cylindrical connecting member outer surface; and
further comprising a fluid-tight sheath covering at least one of said apertures in said first open-cylinder member and said first cylindrical connecting member.

4. The frame of claim 1, further comprising:
a second open-cylinder member with an outer surface defining a circumferential arc length, first and second opposing end portions, and a connection location, wherein
said circumferential arc length is at most equal to the anatomical vessel inner circumference, wherein
said second open-cylinder member is adapted for placement within the lumen of the anatomical vessel with a substantial portion of said second open-cylinder member outer surface contacting the anatomical vessel inner surface, wherein
said second open-cylinder member contacts said first open-cylinder member to form at least one closed-cylinder region, wherein
said connection location is nearer to said first opposing end than said second opposing end; and
a second cylindrical connecting member connected to said second open-cylinder member at said connection location, said second cylindrical connecting member adapted for placement within the lumen of a second connecting branch vessel with a substantial portion of said second cylindrical connecting member outer surface contacting the second connecting branch vessel inner surface.

5. The frame of claim 4, wherein
said second open-cylinder member further comprises at least one aperture through said second open-cylinder member outer surface;
said second cylindrical connecting member further comprises at least one aperture through said second cylindrical connecting member outer surface; and
further comprising a fluid-tight sheath covering at least one of said apertures in said second open-cylinder member and said second cylindrical connecting member.

6. The frame of claim 5, wherein said first open-cylinder member and said second open-cylinder member are adapted to connect to a separate stent graft with a fluid-tight seal.

7. A stent for the anatomical vessels surrounding a junction between a first anatomical vessel and at least a second anatomical vessel, comprising:
a first open-cylinder stent portion with an outer surface, wherein said first open-cylinder stent portion is adapted for placement within the lumen of the first anatomical vessel with said outer surface substantially contiguous with the first anatomical vessel inner surface.

8. The stent of claim 7, wherein said first open-cylinder stent portion defines a first arc length, wherein said first arc length is less than the circumference of the first anatomical vessel inner surface.

9. The stent of claim 7, further comprising a fluid-tight cover, wherein said first open-cylinder stent portion defines a plurality of apertures, and said fluid-tight cover spans at least one said apertures.

10. The stent of claim 7, wherein a portion of said first open-cylinder stent portion allows bodily fluid to flow through said stent portion.

11. The stent of claim 7, wherein said first open-cylinder outer surface is convex when outside the lumen of the first anatomical vessel.

12. The stent of claim 7, further comprising:
a first lateral cylindrical member with an outer surface, said first lateral cylindrical member connected to said first open-cylinder stent portion, wherein said first lateral cylindrical member is adapted for placement within a lumen of the second anatomical vessel with said first lateral cylindrical member outer surface substantially contiguous with the first anatomical vessel inner surface.

13. The stent of claim 12, wherein said first lateral cylindrical member is an open-cylinder.

14. The stent of claim 12, wherein said first open-cylinder stent portion and said first lateral cylindrical member are comprised of a form-returning material.

15. The stent of claim 14, wherein said first open-cylinder stent portion and said first lateral cylindrical member are comprised of a shape memory alloy.

16. The stent of claim 12, wherein said first lateral cylindrical member defines a longitudinal axis along the length of said first lateral cylindrical member, wherein said first lateral cylindrical member is movable relative to said first open-cylinder stent portion such that said longitudinal axis remains substantially within a eighty degree (80°) cone, wherein said cone is stationary in relation to said first open-cylinder stent portion with the cone vertex oriented toward the outer surface of said first open-cylinder stent portion.

17. The stent of claim 16, wherein said cone defines a main axis, wherein said cone main axis is perpendicular to said first open-cylinder stent portion outer surface at the point where said main axis intersects said first open-cylinder stent portion outer surface.

18. The stent of claim 12, wherein said first open-cylinder stent portion and said first lateral cylindrical member are adapted to be collapsed for delivery with a body introduction device.

19. The stent of claim 12, wherein said first open-cylinder stent portion is adapted to exert pressure on the first anatomical vessel inner surface and said first lateral cylindrical member is adapted to exert pressure on the second anatomical vessel inner surface, wherein the exerted pressures act to center said first open-cylinder stent portion and said first lateral cylindrical member in the junction between the first and second anatomical vessels.

20. The stent of claim 12, further comprising:
a second open-cylinder stent portion with at least one aperture and an outer surface, wherein said second open-cylinder stent portion is adapted for placement within the lumen of the first anatomical vessel with said outer surface substantially contiguous with the first anatomical vessel inner surface and in overlapping relation with said first open-cylinder stent portion; and
a second lateral cylindrical member with at least one aperture and an outer surface, said second lateral cylindrical member connected to said second open-cylinder stent portion, wherein said second lateral cylindrical member is adapted for placement within the lumen of a third anatomical vessel with said second lateral cylindrical member outer surface substantially contiguous with the third anatomical vessel inner surface.

21. The stent of claim 20, further comprising:
a fluid-tight cover spanning at least one said aperture in at least one of said second open-cylinder stent portion and said second lateral cylindrical member.

22. The stent of claim 20, wherein said first open-cylinder stent portion defines a first arc length and said second open-cylinder stent portion defines a second arc length, and wherein the sum of said first and second arc lengths is at least equal to the inner circumference of the first anatomical vessel.

23. The stent of claim 20, wherein said second lateral cylindrical member is an open-cylinder.

24. The stent of claim 20, wherein said first and second open-cylinder stent portions and said first and second lateral cylindrical members are comprised of a form-returning material.

25. The stent of claim 24, wherein said first and second open-cylinder stent portions and said first and second lateral cylindrical members are comprised of a shape memory alloy.

26. The stent of claim 20, further comprising:
a third open-cylinder stent portion with at least one aperture and an outer surface, wherein said third open-cylinder stent portion is adapted for placement within the lumen of the first anatomical vessel with said outer surface substantially contiguous with the first anatomical vessel inner surface and in overlapping relation with at least one of the first and second open-cylinder stent portions;
a third lateral cylindrical member with at least one aperture and an outer surface, said third lateral cylindrical member connected to said third open-cylinder stent portion, wherein said third lateral cylindrical member is adapted for placement within the lumen of a fourth anatomical vessel with said third lateral cylindrical member outer surface substantially contiguous with the fourth anatomical vessel inner surface.

27. A method for grafting a region between a reference blood vessel and at least one branching blood vessel, comprising:
collapsing a first open-cylinder stent portion adapted for placement within the lumen of the reference blood vessel;
placing the collapsed first open-cylinder stent portion within the lumen of at least one of the reference blood vessel and a first branching blood vessel; and
expanding the first open-cylinder stent portion, wherein said expanding results in a substantial portion of the first open-cylinder stent portion contacting the reference blood vessel inner surface.

28. The method of claim 27, further comprising:
collapsing a first lateral stent portion attached to the first open-cylinder stent portion, the first lateral stent portion adapted for placement within the lumen of the first branching blood vessel, and wherein the first lateral stent portion is cylindrical;
placing the collapsed first later stent portion within the lumen of at least one of the reference and first branching blood vessels; and
expanding the first lateral stent portion, wherein said expanding result in a substantial portion of the first lateral stent portion contacting the first branching blood vessel inner surface.

29. The method of claim 28 further comprising:
self-aligning the first open-cylinder stent portion with the lumen of the reference blood vessel and the first lateral stent portion within the lumen of the first branching blood vessel, said self-aligning performed by at least one of the first open-cylinder stent portion exerting force on the reference blood vessel inner surface and the first lateral stent portion exerting force on the first branching blood vessel inner surface.

30. The method of claim 28, further comprising:
collapsing a second open-cylinder stent portion and a second lateral stent portion attached to the second open-cylinder stent portion, the second open-cylinder stent portion adapted for placement within the lumen of the reference blood vessel, the second lateral stent portion adapted for placement within the lumen of a second branching blood vessel, and wherein the second lateral stent portion is cylindrical;
placing the collapsed second open-cylinder stent portion and collapsed second lateral stent portion within the lumen of at least one of the reference, first branching and second branching blood vessels; and
expanding the second open-cylinder stent portion and the second lateral stent portion, wherein said expanding results in a substantial portion of the second open-cylinder stent portion contacting the reference blood vessel inner surface, the second open-cylinder stent portion overlapping the first open-cylinder stent portion, and a substantial portion of the second lateral stent portion contacting the second branching blood vessel inner surface.

31. The method of claim 30, wherein said expanding the collapsed second open-cylinder stent portion results in the first and second open-cylinder stent portions together contacting at least one circumferential ring of the reference blood vessel.

32. The method of claim 30 further comprising:
self-aligning the second open-cylinder stent portion with the lumen of the reference blood vessel and the second lateral stent portion within the lumen of the second branching blood vessel, said self-aligning performed by at least one of the second open-cylinder stent portion exerting force on the reference blood vessel inner surface and the second lateral stent portion exerting force on the second branching blood vessel inner surface.

33. The method of claim 30, further comprising:
collapsing a third open-cylinder stent portion and a third lateral stent portion attached to the third open-cylinder stent portion, the third open-cylinder stent portion adapted for placement within the lumen of the reference blood vessel, the third lateral stent portion adapted for placement within the lumen of a third branching blood vessel, and wherein the third lateral stent portion is cylindrical;
placing the collapsed third open-cylinder stent portion and collapsed third lateral stent portion within the lumen of at least one of the reference, first branching, second branching, or third branching blood vessel; and
expanding the third open-cylinder stent portion and the third lateral stent portion, wherein said expanding results in a substantial portion of the third open-cylinder stent portion contacting the reference blood vessel inner surface, the third open-cylinder stent portion overlapping the first and second open-cylinder stent portions, and a substantial portion of the third lateral stent portion contacting the third branching blood vessel inner surface.

34. The method of claim 33, wherein said expanding the collapsed third open-cylinder stent portion results in the first, second and third open-cylinder stent portions together contacting at least one circumferential ring of the reference blood vessel.

35. The method of claim 33 further comprising:
self-aligning the third open-cylinder stent portion with the lumen of the reference blood vessel and the third lateral stent portion within the lumen of the third branching blood vessel, said self-aligning performed by at least one of the third open-cylinder stent portion exerting force on the reference blood vessel inner surface and the third lateral stent portion exerting force on the third branching blood vessel inner surface.
